(19) United States
(12) Patent Application Publication
(10) Pub. No.: US 2024/0091182 A1
(43) Pub. Date: Mar. 21, 2024

(54) LOW DOSE, SUSTAINED RELEASE FORMULATION FOR ALLEVIATING SYMPTOMS CAUSED BY INCREASED LEVELS OF DOPAMINE AND NOREPINEPHRINE

(71) Applicant: Francis Peter Halas, Sea Girt, NJ (US)

(72) Inventor: Francis Peter Halas, Sea Girt, NJ (US)

(21) Appl. No.: 18/369,247

(22) Filed: Sep. 18, 2023

Related U.S. Application Data
(60) Provisional application No. 63/407,207, filed on Sep. 16, 2022.

(51) Int. Cl. A61K 31/195; A61K 31/496; A61K 31/519

(52) U.S. Cl. CPC A61K 31/195; A61K 31/496; A61K 31/519

(57) ABSTRACT

Detailed herein are formulation(s) for alleviating symptoms of autism spectrum disorder as well as other indications. In at least one embodiment, there is described a low dose, sustained release formulation comprising alpha-methyl-L-tyrosine (AMT or AMPT). Such a low dose, sustained release formulation provides a less regimented treatment regimen as well as minimizes potential adverse side effects.

### CROSS-REFERENCE TO RELATED APPLICATIONS

This application claims priority under 35 USC 119(e) to U.S. provisional application 63/407,207 filed Sep. 16, 2022, the entirety of which is incorporated by reference.

### FIELD OF THE INVENTION

The field of the embodiments of the present application relate to formulations for alleviating symptoms associated with, but not limited to anxiety, post traumatic stress disorder (PTSD), hypertension, cytochrome storm, insulin resistance disorders, inflammation, mania, autism spectrum disorder (ASD), and aging via inhibition of dopamine and/or norepinephrine in a patient in need. In particular, described herein are low dose, sustained release formulations comprising alpha-methyl-L-tyrosine (AMT or AMPT) useful for alleviating symptoms of ASD.

### BACKGROUND OF THE INVENTION

Autism and Post Traumatic Stress disorder (PTSD) are characterized by either having too many neurons and synapses or having neurons and synapses that are overly sensitized, respectively. Characteristics of these conditions include excess dopamine and norepinephrine activity. Sensory inputs, memories and thoughts may be too intense and aversive, thereby causing or facilitating these conditions.

Autism spectrum disorder (“autism” or “ASD”) is a neurological and developmental disorder that affects how individuals interact with others, learn, behave, and communicate. ASD is known as a “spectrum” disorder as there is a large degree of variation in the type and severity of symptoms exhibited by those afflicted with ASD. Although ASD can be diagnosed at any age, it is commonly described as a “developmental disorder” as symptoms generally appear within the first two years of life.

There are currently 5.5 million adults and 2 million children in the United States (80 million adults and 20 million children worldwide) that fall somewhere on the autism spectrum. One in 36 8 year old children have been diagnosed to have autism, with males outnumbering females by a 4 to 1 ratio. 30% of children diagnosed with autism are non-verbal into their adulthoods. People with autism have higher mortality rates (especially by suicide) with many taking their lives by violent means. The highest rates occur in Massachusetts, California, Connecticut and New Jersey in the United States and in the United Kingdom, Sweden, Qatar, and the United Arab Emirates worldwide. According to the Diagnostic and Statistical Manual of Mental Disorders (DSM-5), people with ASD often have: difficulty with communication and interaction with other people, restricted interests and repetitive behaviors, and varying symptoms that affect their ability to function in social and other aspects of life.

People of all genders, races, ethnicities, and economic backgrounds can be diagnosed with ASD. Although ASD can be a lifelong disorder, treatments and services can improve a person's symptoms and daily functioning. However, there is currently no one standard treatment for ASD.

Due to the wide ranging symptoms experienced by the general populous afflicted with ASD, the most effective therapies and interventions are often different for each person. That being said, many individuals with ASD respond most favorably to highly structured and specialized programs. Research shows that early diagnosis and interventions, such as during preschool or before, are most likely to have significant positive effects on symptoms. Common treatments for aspects of ASD may include, but are not limited to, behavioral management therapy, cognitive behavior therapy, early intervention, educational and school-based therapies, joint attention therapy, medication treatment, nutritional therapy, occupational therapy, parent-mediated therapy, physical therapy, social skills training, and speech-language therapy.

In some cases, treatment can greatly reduce symptoms and help people with autism with daily activities. The present application and its embodiments aim to alleviate the symptoms of ASD while minimizing treatment regimens and adverse side effects.

PTSD also has a significant impact on the United States and Worldwide populations. Estimates of the number of individuals in the United States at any time that have some degree of PTSD at any time include 10 million women and 5 million men. About 6% of the total population will experience PTSD in their lifetime while 11% of American women will have PTSD sometime in their lifetime. 8% of adolescent females will develop some level of PTSD and 75% of women that have been sexually assaulted experience some level of PTSD. Moreover, 10-30% of veterans from the gulf and Iraq wars have experienced some level of PTSD. Sadly, 15% of veterans that experience PTSD have attempted suicide.

The annual economic burden of ASD and PTSD have been estimated to be about $450 billion for ASD and $300 billion per year in the United States meaning that these two dopamine related disorders have a combined economic impact of three quarters of a trillion dollars annually. When one factors in the worldwide populations, the economic impact is substantially larger.

### REVIEW OF RELATED TECHNOLOGY

U.S. Pat. No. 9,962,336 pertains to a method for preparing a stable extended release suspension composition comprising multiple coated cores of an active ingredient by using a suspension base, wherein the suspension base ensures substantially similar in-vitro dissolution release profile of the active ingredient upon storage of the suspension compositions for at least seven days.

U.S. Pat. No. 8,841,486 pertains to compositions including diastereomers in substantially diastereomerically pure form and enantiomers in substantially enantiomerically pure form, and processes for preparing them and converting them to metyrosine.

U.S. Patent Application Publication 2021/0283083 pertains to the use of a compound that can inhibit tyrosine hydroxylase, such as alpha-methyl-p-tyrosine (AMPT), for the prevention or treatment of aortic aneurysm, preferably of the abdominal aortic.

Thus, various treatments for ASD are commonly known in the art. However, the formulations and methods of treatment are materially different from the present application. The known formulations and methods fail to address the problems taught herein by the present disclosure. At least one embodiment of this invention will be described in more detail herein.

### SUMMARY OF THE INVENTION

In general, the present application and its embodiments provide for formulations for a low dosage, sustained release composition effective for alleviating symptoms associated with, but not limited to anxiety, post traumatic stress disorder (PTSD), hypertension, cytokine storm, insulin resistance disorders, inflammation, mania, autism spectrum disorder (ASD), and aging via inhibition of dopamine and/or norepinephrine in a patient in need.

In one aspect of the present invention there is a composition for alleviating symptoms of autism spectrum disorder (ASD), the composition comprising: a therapeutically effective amount of a tyrosine hydroxylase inhibitor, wherein the therapeutically effective amount is in a range of 0.1 mg to 300 mg. In a variation, the therapeutically effective amount may depend on the weight of the patient so that those patients that are heavier are administered a higher dose. For example, a patient may be administered the composition at a range of 0.1-5.0 mg of the therapeutically effective amount of the compound/kg mass of the patient. In one embodiment of the present application the tyrosine hydroxylase inhibitor is metyrosine.

In one embodiment of the present application the therapeutically effective amount is in a range of 50 mg to 100 mg.

In one embodiment of the present application the therapeutically effective amount is 100 mg.

In one embodiment of the present application the metyrosine is alpha-methyl-L-tyrosine.

In one embodiment of the present application the composition is an extended release composition.

In one embodiment of the present application the composition is a tablet or transcutaneous patch.

In another aspect of the present application there is a composition to inhibit dopamine and/or norepinephrine, the composition comprising a therapeutically effective amount of a tyrosine hydroxylase inhibitor.

In one embodiment of the present application the composition is effective for alleviating symptoms associated with anxiety, post traumatic stress disorder (PTSD), hypertension, cytochrome storm, insulin resistance disorders, inflammation, mania, autism spectrum disorder (ASD), and aging.

In yet another aspect of the present application there is a method of alleviating symptoms of autism spectrum disorder (ASD), the method comprising the steps of: administering a therapeutically effective amount of a tyrosine hydroxylase inhibitor to a patient in need, wherein the therapeutically effective amount is in a range of 0.1 mg to 300 mg.

In one embodiment of the present application the therapeutically effective amount is administered four times per every 24 hours.

In one embodiment of the present application the tyrosine hydroxylase inhibitor is metyrosine.

In one embodiment of the present application the therapeutically effect amount is in a range of 50 mg to 100 mg.

In one embodiment of the present application the therapeutically effective amount is 100 mg.

In one embodiment of the present application the metyrosine is alpha-methyl-L-tyrosine.

In one embodiment of the present application the composition is administered orally.

In one embodiment of the present application the composition is administered transcutaneously.

### DETAILED DESCRIPTION OF THE INVENTION

Reference will now be made in detail to each embodiment of the present invention. Such embodiments are provided by way of explanation of the present invention, which is not intended to be limited thereto. In fact, those of ordinary skill in the art may appreciate upon reading the present specification that various modifications and variations can be made thereto.

Although this invention and its embodiments have been described with a certain degree of particularity, it is to be understood that the present disclosure has been made only by way of illustration and that numerous changes in the details of construction and arrangement of parts may be resorted to without departing from the spirit and the scope of the invention.

As referred to herein, unless stated otherwise, all compositional amount or percentages are by weight of the total composition, unless otherwise specified.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

In certain embodiments, when the term “about” or “approximately” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%. In certain embodiments, the term “about” is used to modify a numerical value above and below the stated value by a variance of 0.1%.

As used herein, the word “include,” and its variants, is intended to be non-limiting, such that recitation of items in a list is not to the exclusion of other like items that may also be useful in the compositions and methods of this technology.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Similarly, the terms “can” and “may” and their variants are intended to be non-limiting, such that recitation that an embodiment can or may comprise certain elements or features does not exclude other embodiments of the present technology that do not contain those elements or features.

As used herein, unless stated otherwise, the term patient refers to a mammal, e.g., a human, mouse, rat, guinea pig, dog, cat, horse, cow, pig, or non-human primate, such as a monkey, chimpanzee, or baboon. The terms “subject” and “patient” are used interchangeably. In certain embodiments, the patient is a human. In certain embodiments, the human is a pediatric human. In certain embodiments, the subject is an adult human.

The terms “effective amount” or “therapeutically effective amount” as used herein, unless stated otherwise, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In some embodiments, the effective amount is a dose that is generally effective in alleviating, reducing, noticeably reducing, or eliminating, symptoms associated with ASD. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

Metyrosine is commonly used to treat high blood pressure in people with certain adrenal gland tumors (e.g., pheochromocytoma). Metyrosine is useful in preventing high blood pressure before and immediately after surgery to remove said tumor. It is also used long-term in people who cannot have the surgery. However, metyrosine is not used for other kinds of high blood pressure.

Metyrosine belongs to a class of compounds known as tyrosine hydroxylase inhibitors or THIs. THIs are a class of drugs that inhibit the production of catecholamines. Catecholamines such as adrenaline, noradrenaline, and dopamine help the body respond to stress or fright, and mainly work by inhibiting an enzyme known as tyrosine hydroxylase.

By inhibiting tyrosine hydroxylase, THIs prevent the production of the aforementioned neurotransmitters. Accordingly, THIs are recognized are being beneficial in conditions with excess release of adrenaline and noradrenaline. The present application and its embodiments, however, have identified the usefulness of THIs, particularly metyrosine, as it relates to alleviating symptoms associated with anxiety, post traumatic stress disorder (PTSD), hypertension, cytochrome storm, insulin resistance disorders, inflammation, mania, autism spectrum disorder (ASD), and aging. Each of the aforementioned indications relate to an increase in dopamine and/or norepinephrine.

Thus, embodiments of the present application are directed to a composition and methods to inhibit dopamine and/or norepinephrine production. Inhibition of such neurotransmitters are, as described herein, useful for alleviating symptoms of the above indications and in particular of autism spectrum disorder (ASD). For any composition contained under the purview of the present application, the active pharmaceutical ingredient or API is metyrosine and in particular alpha-methyl-L-tyrosine. The amount of the metyrosine present in the composition may vary depending on a variety of factors including but not limited to the patient's gender and age, indication to be treated, extended or immediate release formulations, frequency of administration of the composition, etc.

It is preferable that the therapeutically effective amount is in a range of 0.1 mg to 300 mg per administration of the composition. However, any amount between and including 0.1 mg and 300 mg is acceptable. For example, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg . . . 300 mg. The amount of the API may be a whole number (e.g., 10 mg) or decimal integer (e.g., 10.5 mg) depending on the needs of the patient and other factors as noted herein. In at least one embodiment, it is more preferable that the composition contains a therapeutically effective amount of metyrosine in a range of 50 mg to 100 mg, and even more particularly 100 mg.

Administration of the composition may be accomplished through traditional means, which include but are not limited to, oral, subcutaneous, intradermal, intramuscular by way of non-limiting example, intramuscular depot, intraperitoneal, intravenous, intranasal, epidural, sublingual, intranasal, intracerebral, intravaginal, transdermal, rectally, by inhalation, or topically, particularly to the ears, nose, eyes, or skin. The mode of administration can be left to the discretion of the practitioner. In most instances, administration results in the release of the compounds described herein or their pharmaceutically acceptable salts into the bloodstream. The preferred mechanism of administration is either orally or by transcutaneous patch. If administered orally, it is preferable that such composition is administered via tablet or capsule containing additional ingredients based on, in part, the desired release profile. However, additional mechanisms of delivery under the purview of this application may include a wide variety of dose forms may include tablets, lozenges, aqueous or oily suspensions, solutions, granules, capsules, powders, pills, pellets, capsules containing liquids, emulsions, syrups, or elixirs, suppositories, sustained-release formulations, or any other form suitable for use.

In a preferred method of alleviating symptoms of autism spectrum disorder (ASD), the method comprising the steps of administering a therapeutically effective amount of a tyrosine hydroxylase inhibitor to a patient in need, wherein the therapeutically effective amount is in a range of 0.1 mg to 300 mg, where the therapeutically effective amount is administered at least four times per twenty four hour period. The number of twenty four hour periods which comprise a treatment protocol will vary as needed.

In addition to the API noted herein, the embodiments of the present compositions can optionally comprise a suitable amount of a pharmaceutically acceptable excipient so as to provide the form for proper administration to the patient. Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipients are sterile when administered to a subject. Water is a useful excipient when the compounds of present invention or their pharmaceutically acceptable salts are administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, sodium chloride, dried skim milk, glycerol, propylene, glycol, water, ethanol, and the like. The present compositions, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference in its entirety.

In an embodiment, the composition of the present invention may be administered as a prodrug so as to overcome barriers in the patient, such as lack of solubility, permeability, stability, presystemic metabolism, or systemic targeting limitations.

In an embodiment, the composition of the present invention may be administered in conjunction with risperidone and/or aripiprazole.

In an embodiment the composition of the present invention and the therapeutically effective agent may be administered as a polymorph and/or in a crystallized form. In a variation, the therapeutically effective agent may be administered as a solvate. In a variation, the therapeutically effective agent may be administered in enantiomerically pure form or a partially pure form. Alternatively, the therapeutically effective agent may be administered as a racemic mix.

Orally administered compositions can comprise one or more agents, for example, sweetening agents such as fructose, aspartame, or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period. Selectively permeable membranes surrounding an osmotically active compound of the invention are also suitable for oral administration. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

Where the compounds described herein or their pharmaceutically acceptable salts can be administered by controlled-release or sustained-release means or by delivery devices that are well known to those of ordinary skill in the art. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions.

Suitable controlled- or sustained-release formulations known to those skilled in the art, including those described herein, can be readily selected for use metyrosine, or a pharmaceutically acceptable salt thereof. In certain embodiments, the embodiments thus provide single unit dosage forms suitable for oral administration such as, but not limited to, tablets, capsules, gel caps, and caplets that are adapted for controlled- or sustained-release.

In certain embodiments, the ingredients of a single unit dosage are supplied either separately or mixed together, for example, as a dry lyophilized-powder or water-free concentrate in a hermetically sealed container such as an ampule or sachet indicating the quantity of active agent. Where the compounds described herein or their pharmaceutically acceptable salts are to be administered by infusion, they can be dispensed, for example, with an infusion bottle containing sterile pharmaceutical grade water or saline. Where the compounds described herein or their pharmaceutically acceptable salts are administered by injection, an ampule of sterile water for injection or saline can be provided so that the ingredients can be mixed prior to administration.

“Controlled release” as used herein is meant to encompass release of API (e.g., metyrosine) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

In certain embodiments, and by way of example only, the composition optionally includes one or more nutraceuticals, selected from the group consisting of vitamin B2 (riboflavin), glucosamine HCl, chlorogenic acid, lipoic acid, catechin hydrate, creatine, acetyl-L-carnitine HCl, vitamin B6, pyridoxine, caffeic acid, naringenin, vitamin B1 (thiamine HCl), baicalein, luteolin, hesperidin, rosmarinic acid, epicatechin gallate, epigallocatechin, vitamin B9 (folic), genistein, methylvanillin, ethylvanillin, silibinin, daidzein, melatonin, rutin hydrate, vitamin A, retinol, vitamin D2 (ergocalciferol), vitamin E (tocopherol), diosmin, menadione (K3), vitamin D3 (cholecalciferol), phloretin, indole-3-carbinol, fisetin, glycitein, chrysin, gallocatechin, vitamin B4 (adenine), vitamin B5 (pantothenic acid), vitamin B7 (biotin), theobromine, resveratrol, epigallocatechin-3-gallate (EGCG), quercetin, ferulic acid, ellagic acid, hesperidin, and protocatechuic acid.

In an embodiment, the present invention relates to treating patients that have dopamine and/or norepinephrine related disorders, for example, ASD and PTSD. The treatment parameters are as discussed herein. During treatment, the patients should be given a dose and the dose can be titrated to the appropriate level by monitoring the patients and looking for relevant changes that may occur to the treated patient. Amongst the changes that should be monitored include any perspective change that might occur with the patient, looking for any physical change of the patient, looking for appropriate chemical change (like the levels of dopamine and/or norepinephrine, looking for appearance change in the patient, looking for metabolism change, looking at the delivery mechanism and whether or not there is a transport change, ascertaining any potency change related to the therapeutically effective agent, looking for any stability change or permeability change that may occur, looking at any clearance change to see how the body processes relevant metabolites to either clear them from the system or compositional changes that may occur. In a variation, the present invention to some extent relates to a method of monitoring changes in an individual that has been treated with the therapeutically effective agent as discussed herein and monitoring the patient for the above enumerated changes. The dose should be titrated so as to obtain more optimal conditions manifested by these changing parameters.

In an embodiment, the present invention relates to a composition for alleviating symptoms of autism spectrum disorder (ASD), the composition comprising:
a therapeutically effective amount of a tyrosine hydroxylase inhibitor,
   wherein the therapeutically effective amount is in a range of 0.1 mg to 300 mg.

In a variation, the tyrosine hydroxylase inhibitor is metyrosine. In a variation, the therapeutically effective amount is in a range of 50 mg to 100 mg. In a variation, the therapeutically effective amount is about 100 mg. In an embodiment, the metyrosine is alpha-methyl-L-tyrosine. In a variation, the composition is an extended release composition. In a variation, the composition is a tablet or transcutaneous patch.

In a variation, the present invention relates to methods for alleviating symptoms of ASD with dosing the same as indicated above. Moreover, the method includes using alpha-methyl-L-typrosine and/or extended release compositions. Further, the method includes having the composition as a tablet or a transcutaneous patch.

In an embodiment, the present invention relates to a method of inhibiting dopamine or norepinephrine, the method comprising administering a therapeutically effective amount of a tyrosine hydroxylase inhibitor to an individual in need thereof. The tyrosine hydroxylase inhibitor includes alpha-methyl-L-tyrosine. In a variation, the method includes administering the therapeutically effective amount as an extended release composition. In a variation, the method allows for its use as a tablet or transcutaneous patch. The method includes alleviating symptoms from maladies/conditions including anxiety, post traumatic stress disorder (PTSD), hypertension, cytokine storm, insulin resistance disorders, inflammation, mania, autism spectrum disorder (ASD), and aging.

In an embodiment, the present invention relates to a composition to inhibit dopamine and/or norepinephrine, the composition comprising a therapeutically effective amount of a tyrosine hydroxylase inhibitor. In a variation, the composition is effective for alleviating symptoms associated with anxiety, post traumatic stress disorder (PTSD), hypertension, cytokine storm, insulin resistance disorders, inflammation, mania, autism spectrum disorder (ASD), and aging.

In an embodiment, the composition and methods include administering the composition along with other medicines. In a variation, the composition and method includes a composition that further comprises risperidone and/or aripiprazole. In a variation, the composition and methods of using the composition have a composition that further comprises one or more of an excipient, an adjuvant, and/or a surfactant.

In an embodiment, the present invention relates to a method of alleviating symptoms of autism spectrum disorder (ASD), the method comprising the steps of:
administering a therapeutically effective amount of a tyrosine hydroxylase inhibitor to a patient in need,
   wherein the therapeutically effective amount is in a range of 0.1 mg to 300 mg.

In a variation, the therapeutically effective amount is administered four times per every 24 hours. In a variation, the therapeutically effective amount is administered once a day, twice a day or three times a day. In a variation, the tyrosine hydroxylase inhibitor is metyrosine. In a variation, the therapeutically effect amount is in a range of 50 mg to 100 mg.

In a variation, the therapeutically effective amount is 100 mg. In a variation, the metyrosine is alpha-methyl-L-tyrosine. In a variation of the method, the composition is administered orally. In a variation, the composition is administered transcutaneously. In a variation, the metyrosine is administered in conjunction with an adjuvant, excipient, and/or surfactant.

In a variation, the compositions and methods of the present invention allow for the therapeutically effective amount of the compounds of the present invention to be administered as a racemate or alternatively, as the pure isomer (or as a mix wherein one of the isomers is present in enantiomeric excess). When the therapeutically effective amount of the compounds of the present invention are administered as a pure isomer it should be understood that lower dosing may be able to be attained. Moreover, any disadvantageous conditions that are caused by the non-active isomer may be mitigated and/or eliminated. In a variation, the biologically active isomer may be administered at a dose that is half the dose of a racemic mixture and one may get the same or potentially a better effect (as side effects may be mitigated or eliminated). In a variation, the racemic mixture or the pure isomer of the compounds of the present invention (e.g., metryrosine) may be administered as slow and/or sustained release compositions. The racemic mix or the pure isomer may be used for any of ASD, PTSD, anxiety, cytokine storm, hypertension, insulin resistance, mania, and/or inflammation.

Although this invention has been described with a certain degree of particularity, it is to be understood that the present disclosure has been made only by way of illustration and that numerous changes in the details of construction and arrangement of parts may be resorted to without departing from the spirit and the scope of the invention.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict between the present explicit disclosure and a document incorporated by reference, the present explicit disclosure shall be the operative disclosure.

What is claimed is:

1. A composition for alleviating symptoms of autism spectrum disorder (ASD), the composition comprising:
a therapeutically effective amount of a tyrosine hydroxylase inhibitor,
wherein the therapeutically effective amount is in a range of 0.1 mg to 300 mg.

2. The composition of claim 1 wherein the tyrosine hydroxylase inhibitor is metyrosine.

3. The composition of claim 1 wherein the therapeutically effective amount is in a range of 50 mg to 100 mg.

4. The composition of claim 3 wherein the therapeutically effective amount is about 100 mg.

5. The composition of claim 2 wherein the metyrosine is alpha-methyl-L-tyrosine.

6. The composition of claim 1 wherein the composition is an extended release composition.

7. The composition of claim 1 wherein the composition is a tablet or transcutaneous patch.

8. A method of inhibiting dopamine and/or norepinephrine in an individual in need thereof, the method comprising administering a therapeutically effective amount of a tyrosine hydroxylase inhibitor to said individual.

9. The method of claim 8 wherein the therapeutically effective amount of a tyrosine hydroxylase inhibitor is administered as a composition, said composition being effective for alleviating symptoms associated with anxiety, post traumatic stress disorder (PTSD), hypertension, cytokine storm, insulin resistance disorders, inflammation, mania, autism spectrum disorder (ASD), and aging.

10. The composition of claim 5, wherein the composition further comprises risperidone and/or aripiprazole.

11. The composition of claim 10, wherein the composition further comprises one or more of an excipient, an adjuvant, and/or a surfactant.

12. A method of alleviating symptoms of autism spectrum disorder (ASD), the method comprising the steps of:
administering a therapeutically effective amount of a tyrosine hydroxylase inhibitor to a patient in need,
wherein the therapeutically effective amount is in a range of 0.1 mg to 300 mg.

13. The method of claim 12 wherein the therapeutically effective amount is administered four times per every 24 hours.

14. The method of claim 12 wherein the tyrosine hydroxylase inhibitor is metyrosine.

15. The method of claim 12 wherein the therapeutically effect amount is in a range of 50 mg to 100 mg.

16. The method of claim 15 wherein the therapeutically effective amount is 100 mg.

17. The method of claim 14 wherein the metyrosine is alpha-methyl-L-tyrosine.

18. The method of claim 12 wherein the composition is administered orally.

19. The method of claim 12 wherein the composition is administered transcutaneously.

20. The method of claim 14, wherein the metyrosine is administered in conjunction with an adjuvant, excipient, and/or surfactant.
